(19) United States
(12) Patent Application Publication
(10) Pub. No.: US 2011/0313275 A1
(43) Pub. Date: Dec. 22, 2011

(54) Method and system for providing magnetic resonance images

(75) Inventors: Felix Güttler, Berlin (DE); Jens Rump, Berlin (DE); Ulf Teichgräber, Berlin (DE)

(73) Assignee: Charite-Universitatsmedizin Berlin

(21) Appl. No.: 12/801,655

(22) Filed: Jun. 18, 2010

(51) Int. Cl. A61B 5/055

(52) U.S. Cl. 600/410

(57) ABSTRACT

An embodiment of the invention relates to a method for providing magnetic resonance images of a foetus or a mother carrying a foetus, the method comprising the steps of:
applying an ultrasonic transceiver to the mother's body and inducing an ultrasonic signal into the mother's and foetus's body;
receiving a returned ultrasonic signal and providing an output signal;
processing the output signal in order to generate a trigger signal indicating the heart activity of the foetus;
triggering a magnetic resonance imaging device taking the trigger signal into account; and
generating magnetic resonance images of the foetus or the mother by using the triggered magnetic resonance imaging device.

### BACKGROUND OF THE INVENTION

The invention relates to a method and system for providing magnetic resonance images of a foetus or a mother carrying a foetus.

Various diagnostic approaches exist for the exploration of foetal heart disease. Today, imaging modalities have an integral part in prenatal diagnostics; i.e. conventional echocardiography and magnetic resonance imaging (MRI). Although MRI shows superior soft tissue contrast, foetal cardiac imaging for prenatal heart function assessment is performed with ultrasonography in clinical routine. This is explained by insufficient visibility of moving structures with MRI.

Problems, which occur when imaging moving organs with MRI, may be avoided by triggering the MRI. However, in vivo foetal cardiac MRI still lacks a feasible triggering concept, which is safe for both foetus and mother and allows reliable and fast detection of foetal heart sounds.

Motion artefacts, which occur when imaging moving organs with

MRI, may be avoided with triggered or fast image acquisition. However, in foetal cardiac imaging, electrocardiographic (ECG) MRI triggering is associated with unjustifiable risks. Foetal ECG requires an cabled electrode to be invasively placed on the foetus's head, through which currents may be induced from the MRI and shock the foetus and the mother.

Foetal cardiac imaging may also be accomplished by reducing the duration of image acquisition with fast gradient systems in combination with sequence optimization and parallel imaging. However, the design of fast gradient systems is limited by the risk of peripheral nervous stimulation, and image quality will typically reduce with faster sequences.

### OBJECTIVE OF THE PRESENT INVENTION

An objective of the present invention is to provide a method and system which delivers images of a foetus or a mother carrying a foetus in a safe manner.

A further objective of the present invention is to provide a method and system which avoids artefacts when capturing images of a foetus or a mother carrying a foetus.

A further objective of the present invention is to provide a method and system which can be carried out without cabled electrodes invasively placed on the foetus's head.

### BRIEF SUMMARY OF THE INVENTION

An embodiment of the invention relates to a method for providing magnetic resonance images of a foetus or a mother carrying a foetus, the method comprising the steps of:
applying an ultrasonic transceiver to the mother's body and inducing an ultrasonic signal into the mother's and foetus's body;
receiving a returned ultrasonic signal and providing an output signal;
processing the output signal in order to generate a trigger signal indicating the heart activity of the foetus;
triggering a magnetic resonance imaging device taking the trigger signal into account; and
generating magnetic resonance images of the foetus or the mother by using the triggered magnetic resonance imaging device.

Preferably, the pictures are captured when the foetus's heart does not beat.

Another embodiment of the invention relates to a system for providing magnetic resonance images of a foetus or a mother carrying a foetus, the system comprising
a magnetic resonance imaging device;
an ultrasonic transceiver having
   a transmitter adapted to provide an ultrasonic signal and adapted to induce the ultrasonic signal into the mother's body; and
   a receiver adapted to receive a returned ultrasonic signal and to provide an output signal;
a trigger device connected to the ultrasonic transceiver, said trigger device being configured to process the output signal and to provide a trigger signal for triggering the magnetic resonance imaging device, said trigger signal indicating the heart activity of the foetus;
wherein said magnetic resonance imaging device is adapted to capture magnetic resonance images taking the trigger signal provided by the trigger device into account.

The ultrasonic sensor may comprise a Doppler ultrasonic sensor unit.

Preferably, the trigger device comprises a filter adapted to reduce the noise comprised in the output signal. For instance, the filter may be a Wiener-filter which is adapted to reduce the noise by comparison of the output signal with an estimation of the desired noiseless output signal.

Alternatively or additionally, the trigger device may comprise a comb filter configured to reduce disturbance frequencies generated by the magnetic resonance imaging device in the output signal.

Alternatively or additionally, the trigger device may comprise a fast Fourier transform unit configured to transform the output signal from the time-domain into the frequency-domain, and an inverse fast Fourier transform unit configured to transform the output signal from the frequency-domain back into the time-domain. In this specific embodiment, a comb filter is preferably arranged between the fast Fourier transform unit and the inverse fast Fourier transform unit.

Alternatively or additionally, the trigger device may comprise a band pass filter configured to reduce the bandwidth of the output signal in the frequency-domain. The bandwidth of the frequencies passing the band pass filter preferably corresponds to the typical spectrum of the foetus's heart sound. The band pass filter is preferably arranged between the fast Fourier transform unit and the inverse fast Fourier transform unit. The bandwidth of the band pass filter preferably ranges between 20 Hz and 180 Hz.

Alternatively or additionally, the trigger device may comprise a mean-value-filter which processes the signal outputted by the inverse fast Fourier transform unit.

Alternatively or additionally, the trigger device may comprise an auto- or cross-correlation function unit.

According to a preferred embodiment, the trigger device comprises
a Wiener-filter configured to reduce the noise by comparison of the output signal with an estimation of the desired noiseless output signal in the time-domain;
a fast Fourier transform unit configured to transform the output signal from the time-domain into the frequency-domain;
an inverse fast Fourier transform unit configured to transform the output signal from the frequency-domain back into the time-domain;
a comb filter configured to reduce disturbance frequencies generated by the magnetic resonance imaging device in the output signal, and to filter the output signal in the frequency-domain, said comb filter being arranged between said fast Fourier transform unit and said inverse fast Fourier transform unit;
a band pass filter configured to reduce the bandwidth of the output signal in the frequency-domain, said band pass filter being arranged between said fast Fourier transform unit and said inverse fast Fourier transform unit, wherein the bandwidth passing the band pass filter corresponds to the typical spectrum of the foetus's heart sound;
a mean-value-filter; and
an auto- or cross-correlation function unit.

The trigger device or the magnetic resonance imaging device may comprise a time-shifter unit in order to start the picture taking process whenever the foetus's heart does not beat.

The transceiver and the trigger device may be connected by a cable or wirelessly by a radio signal.

### BRIEF DESCRIPTION OF THE DRAWINGS

In order that the manner in which the above-recited and other advantages of the invention are obtained will be readily understood, a more particular description of the invention briefly described above will be rendered by reference to specific embodiments thereof which are illustrated in the appended drawings. Understanding that these drawings depict only typical embodiments of the invention and are therefore not to be considered to be limiting of its scope, the invention will be described and explained with additional specificity and detail by the use of the accompanying drawings in which

FIG. 1 shows a first exemplary embodiment of an inventive system;

FIG. 2 shows an unfiltered output signal of a transceiver in an exemplary fashion;

FIG. 3 shows a filtered output signal of a transceiver in an exemplary fashion;

FIG. 4 shows an exemplary embodiment of a trigger device for the system of FIG. 1; and

FIG. 5 shows a second exemplary embodiment of an inventive system.

### DETAILED DESCRIPTION OF THE PREFERRED EMBODIMENT

The preferred embodiment of the present invention will be best understood by reference to the drawings, wherein identical or comparable parts are designated by the same reference signs throughout.

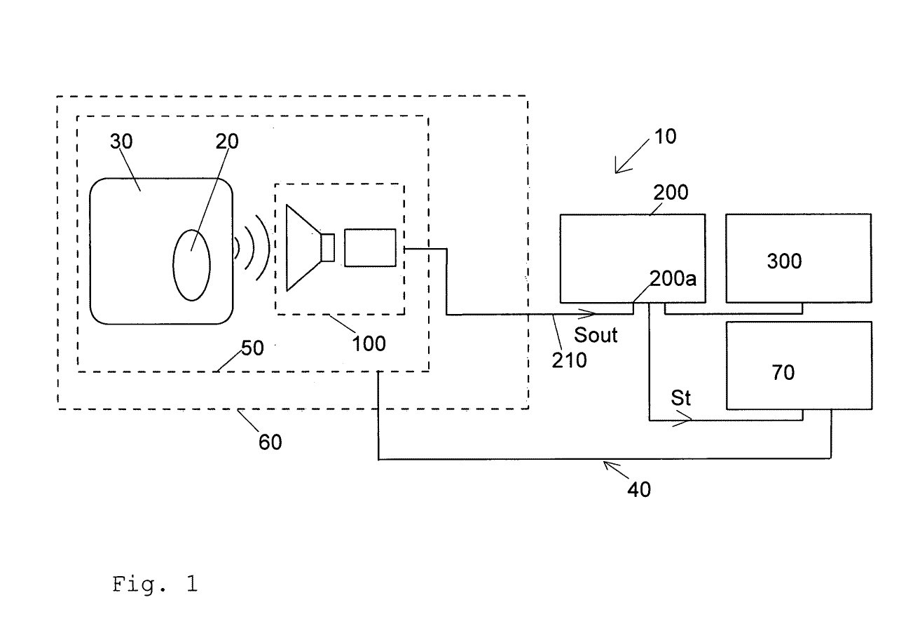

It will be readily understood that the present invention, as generally described and illustrated in the figures herein, could vary in a wide range. Thus, the following more detailed description of the exemplary embodiments of the present invention, as represented in FIGS. 1-5, is not intended to limit the scope of the invention, as claimed, but is merely representative of presently preferred embodiments of the invention.

FIG. 1 shows an exemplary embodiment of a system 10 for providing magnetic resonance images of a foetus 20 or a mother 30 carrying the foetus.

The system 10 comprises a magnetic resonance imaging device 40 comprising a picture taking unit 50 arranged inside a shielded room 60, and a controller unit 70 arranged outside the shielded room 60.

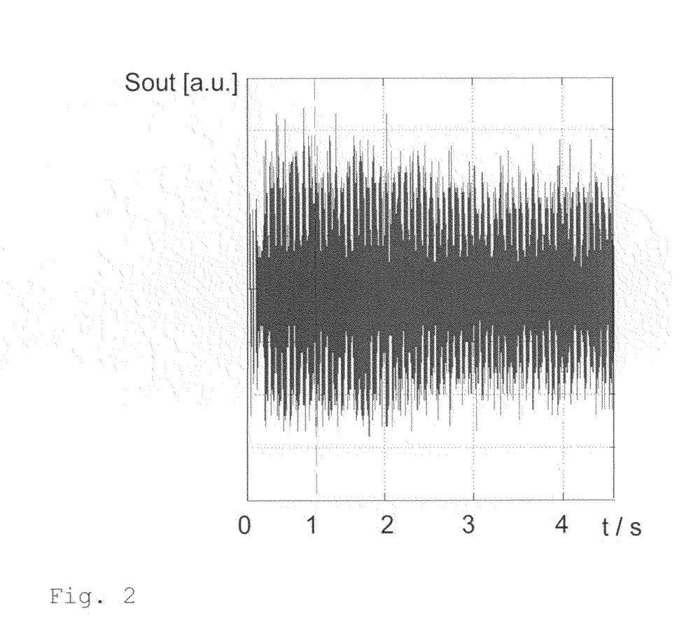

The system 10 further comprises an ultrasonic transceiver 100 having a transmitter adapted to provide an ultrasonic signal and adapted to induce the ultrasonic signal into the mother's body 30. The ultrasonic transceiver 100 further comprises a receiver adapted to receive a returned ultrasonic signal and to provide a preferably sampled output signal Sout. The output signal Sout is depicted in FIG. 2 in an exemplary fashion.

Furthermore, the system 10 comprises a trigger device 200 arranged outside the shielded room 60 and connected to the ultrasonic transceiver 100 via a cable 210. The cable 210 may be a shielded and electrically conductive cable which electrically transmits the output signal Sout. Alternatively, the cable 210 may be an optical cable such as an optical fiber which transmits the output signal Sout in an optical manner.

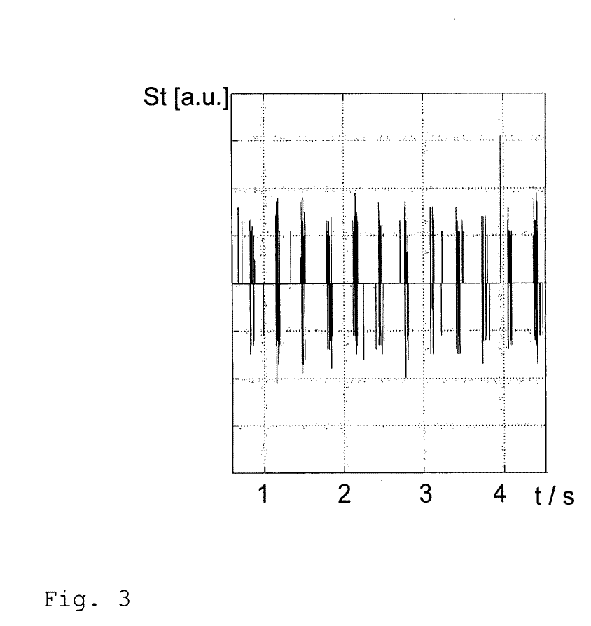

The trigger device 200 processes the output signal Sout and provides a trigger signal St for triggering the controller unit 70 and thus the entire magnetic resonance imaging device 40. The trigger signal St is depicted in FIG. 3 in an exemplary fashion. It can be seen that the trigger signal St indicates the heart activity and the heart beat of the foetus.

The controller unit 70 controls the picture taking unit 50 according to the trigger signal Sout provided by trigger device 200 and thus avoids artefacts in the pictures captured. Preferably, the pictures are taken when the foetus's heart does not beat.

The system 10 may further comprise a foetal monitor 300 showing the signal provided by the ultrasonic transceiver 100. Preferably the foetal monitor 300 indicates the foetal heartbeats as well as the mother's uterine contractions.

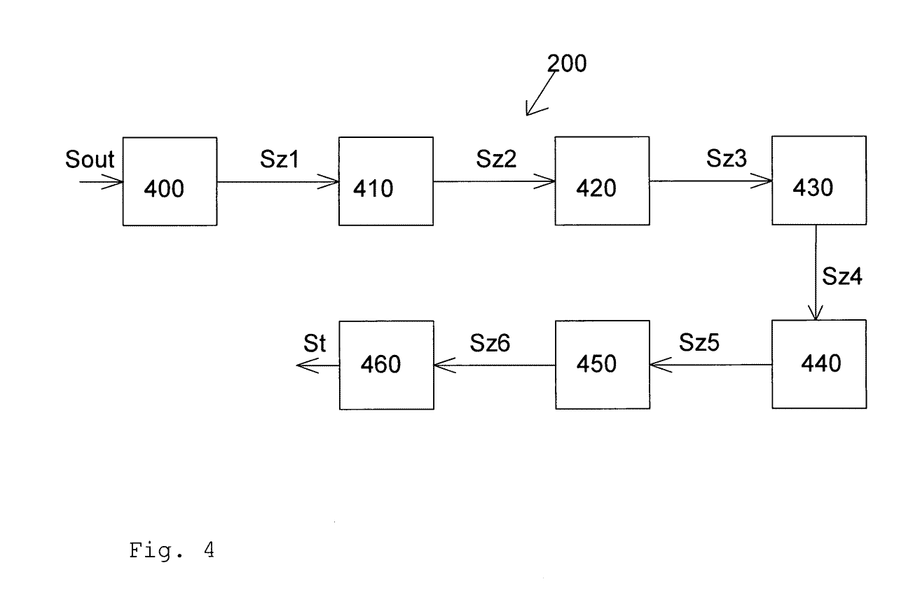

FIG. 4 shows an exemplary embodiment of the trigger device 200. The trigger device 200 comprises a plurality of digital units which process the sampled output signal Sout.

A digital Wiener-filter 400 may be connected to an input 200a of trigger device 200 (see FIG. 1). The Wiener-filter 400 applies the Wiener-Kolmogorov filtering theory and reduces the noise by comparison of the sampled output signal Sout with an estimation of the desired noiseless output signal in the time-domain.

The filtered signal Sz1 provided by Wiener-filter 400 is inputted into a digital fast Fourier transform unit 410 which transforms the filtered signal Sz1 from the time-domain into the frequency-domain.

The transformed signal Sz2 reaches a digital band pass filter 420. The band pass filter 420 transmits frequencies corresponding to the typical spectrum of the foetus's heart sound and blocks the other frequencies. Preferably, the spectrum for which the band pass filter 420 is transparent, ranges from 10 Hz to 200 Hz, for instance from 20 Hz to 180 Hz. The output signal of the band pass filter 420 is marked by reference sign Sz3 in FIG. 4.

The signal Sz3 of the band pass filter 420 then enters a digital comb filter 430. The Comb filter 430 reduces disturbance frequencies generated by the picture taking unit 50 of the magnetic resonance imaging device 40. To this end, the comb filter 430 filters the signal Sz3 in the frequency-domain. The frequencies fcomb filtered or blocked by the comb filter 430 are preferably given by the following equation:

fcomb=Q/TR

wherein TR is the repetition frequency of the picture taking unit 50, i.e. the repetition frequency according to which the pictures are captured. Q is a set of natural numbers {1, 2, 3, . . . }.

The filtered signal which exits the comb filter 430 is marked by reference sign Sz4 in FIG. 4.

The trigger device 200 further comprises a digital inverse fast Fourier transform unit 440 which transforms the output signal Sz4 of the comb filter 430 from the frequency-domain back into the time-domain.

The retransformed signal Sz5 reaches a digital mean-value-filter 450 which reduces remaining noise carried by signal Sz4.

Preferably, the trigger device 200 further comprises an auto- or cross-correlation function unit 460 which filters the heart beat signal from residual signals. An autocorrelation function and/or an cross-correlation function can be calculated in time domain or in frequency domain.

For instance, the autocorrelation function can be calculated as follows:

S(f)=x*(f)·x(f)

wherein x(f) describes signal Sz6 in the frequency domain and x*(f)the conjugate complex function thereof. The autocorrelation function may then be written as:

$${R}_{1} ( τ ) = {\int }_{- ∞}^{∞} S ( f ) \cdot {e}^{i 2 π f τ} d f$$

The duration T₁ of the foetus's heart beat (heart cycle) results from a determination of the maximum of the autocorrelation function R₁(τ):

T₁=max(R₁(τ)) for τ≦0.25 s

Based on the value of T₁ the auto- or cross-correlation function unit 460 generates the signal St that may be used to trigger the controller unit 70 and thus the picture taking process.

In the manner described above in an exemplary fashion, pictures of the foetus and/or the mother can be taken at the appropriate moments in view of the foetal heart beat. For instance, the pictures can be captured in time frames when the foetus's heart does not interact, i.e. in time frames when the foetus's heart does not beat and thus does not influence the picture's quality. For instance, the pictures may be captured time-shifted to the trigger signal St in order to guarantee that the pictures are taken when the foetus's heart does not beat.

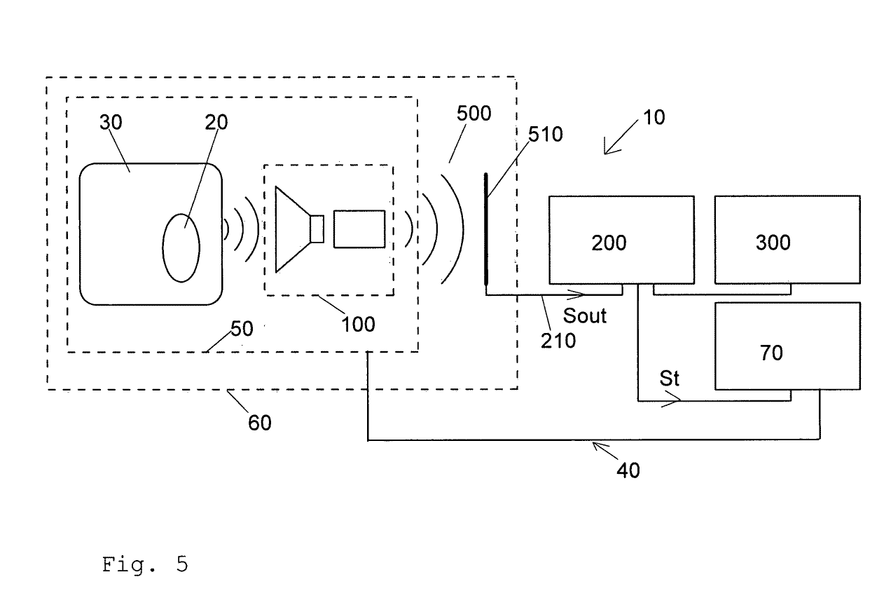

FIG. 5 shows a further exemplary embodiment of a system 10 for providing magnetic resonance images of a foetus 20 or a mother 30 carrying the foetus. The system 10 of FIG. 5 differs from system 10 of FIG. 1 in that the output signal Sout of the receiver is transmitted wireless via a radio signal 500 from the inside of the picture taking unit 50 to an antenna 510 arranged outside the picture taking unit 50 and inside the shielded room 60. The cable 210 may be used to transmit the output signal Sout from the antenna 510 to the trigger device 200 in an electrical or optical manner.

The frequency of radio signal 500 is preferably outside the frequency range of the magnetic resonance imaging device 40 in order to avoid interference. For instance, for the radio signal 500, a frequency of about 900 Mhz may be used. Alternatively, a frequency range between 2.4 GHz und 2.5 GHz, e. g. according to or in view of Bluetooth standard IEEE 802.15.1 or WLAN standard IEEE 802.11 (WLAN: Wireless Local Area Network) may be used. It is also possible to use higher frequencies, for instance between 5 GHz und 6 GHz, e. g. according to or in view of the WLAN standard.

In the system 10 shown in FIGS. 1 and 5, the trigger device 200 or the magnetic resonance imaging device 40 may comprise a time-shifter unit for triggering the picture taking process time-shifted relative to the foetus's heart beat.

### REFERENCE SIGNS

10 system

20 foetus

30 mother

40 magnetic resonance imaging device

50 picture taking unit

60 shielded room

70 controller unit

100 ultrasonic transceiver

200 trigger device

210 cable

300 foetal monitor

400 Wiener-filter

410 fast Fourier transform unit

420 band pass filter

430 comb filter

440 inverse fast Fourier transform unit

450 mean-value-filter

460 auto- or cross-correlation function unit

500 radio signal

510 antenna

Sout signal

Sz1-Sz7 signal

St signal

1. Method for providing magnetic resonance images of a foetus or a mother carrying a foetus, the method comprising the steps of:
applying an ultrasonic transceiver to the mother's body and inducing an ultrasonic signal into the mother's and foetus's body;
receiving a returned ultrasonic signal and providing an output signal;
processing the output signal in order to generate a trigger signal indicating the heart activity of the foetus;
triggering a magnetic resonance imaging device taking the trigger signal into account; and
generating magnetic resonance images of the foetus or the mother by using the triggered magnetic resonance imaging device.

2. Method of claim 1 wherein the pictures are captured time-shifted to the foetus's heart beat.

3. System for providing magnetic resonance images of a foetus or a mother carrying a foetus, the system comprising
a magnetic resonance imaging device;
an ultrasonic transceiver having
a transmitter adapted to provide an ultrasonic signal and adapted to induce the ultrasonic signal into the mother's body; and
a receiver adapted to receive a returned ultrasonic signal and to provide an output signal;
a trigger device connected to the ultrasonic transceiver, said trigger device being configured to process the output signal and to provide a trigger signal for triggering the magnetic resonance imaging device, said trigger signal indicating the heart activity of the foetus;
wherein said magnetic resonance imaging device is adapted to capture magnetic resonance images taking the trigger signal into account.

4. System of claim 3 wherein said ultrasonic sensor is a Doppler ultrasonic sensor.

5. System of claim 3 wherein said trigger device comprises a filter adapted to reduce the noise comprised in the output signal.

6. System of claim 5 wherein said filter is a Wiener-filter which is adapted to reduce the noise by comparison of the output signal with an estimation of the desired noiseless output signal.

7. System of claim 3 wherein said trigger device comprises a comb filter configured to reduce disturbance frequencies generated by the magnetic resonance imaging device in the output signal.

8. System of claim 3 wherein said trigger device comprises
a fast Fourier transform unit configured to transform the output signal from the time-domain into the frequency-domain; and
an inverse fast Fourier transform unit configured to transform the output signal from the frequency-domain back into the time-domain.

9. System of claim 6
wherein said trigger device comprises a comb filter configured to comb-filter the output signal in the frequency-domain; and
wherein said comb filter is arranged between said fast Fourier transform unit and an inverse fast Fourier transform unit.

10. System of claim 8 wherein said trigger device comprises a Wiener-filter adapted to reduce the noise by comparison of the output signal with an estimation of the desired noiseless output signal, said Wiener-filter being arranged between the trigger device's input interface and said fast Fourier transform unit.

11. System of claim 8
wherein said trigger device comprises a band pass filter configured to reduce the bandwidth of the output signal in the frequency-domain;
wherein the bandwidth passing the band pass filter corresponds to the typical spectrum of the foetus's heart sound; and
wherein said band pass filter is arranged between said fast Fourier transform unit and said inverse fast Fourier transform unit.

12. System of claim 11 wherein the bandwidth passing the band pass filter includes the frequency range between 20 Hz and 180 Hz.

13. System of claim 8 wherein the trigger device comprises a mean-value-filter which processes the signal outputted by the inverse fast Fourier transform unit.

14. System of claim 3 wherein the trigger device comprises a auto- or cross-correlation function unit.

15. System of claim 3 wherein said trigger device comprises
a Wiener-filter configured to reduce the noise by comparison of the output signal with an estimation of the desired noiseless output signal in the time-domain;
a fast Fourier transform unit configured to transform the output signal from the time-domain into the frequency-domain;
an inverse fast Fourier transform unit configured to transform the output signal from the frequency-domain back into the time-domain;
a comb filter configured to reduce disturbance frequencies generated by the magnetic resonance imaging device in the output signal, and to filter the output signal in the frequency-domain, said comb filter being arranged between said fast Fourier transform unit and said inverse fast Fourier transform unit;
a band pass filter configured to reduce the bandwidth of the output signal in the frequency-domain, said band pass filter being arranged between said fast Fourier transform unit and said inverse fast Fourier transform unit, wherein the bandwidth passing the band pass filter corresponds to the typical spectrum of the foetus's heart sound;
a mean-value-filter; and
an auto- or cross-correlation function unit.

16. System of claim 3 wherein said trigger device or the magnetic resonance imaging device comprises a time-shifter unit.

17. System of claim 3 wherein the transceiver is wirelessly connected to the trigger device.
